SEQUENCE LISTING (3 sequences)

SEQ ID NO: 1 - DNA, 18 bp, Artificial Sequence
  forward primer
tgtatgaagaaggcttcg

SEQ ID NO: 2 - DNA, 19 bp, Artificial Sequence
  reverse primer
aaaggtattaactttactc

SEQ ID NO: 3 - DNA, 68 bp, Artificial Sequence
  TaqMan probe
tgtatgaagaaggcttcgggttgtaaagtactttcagcggggaggaagggagtaaagttaataccttt

(19) United States
(12) Patent Application Publication
(10) Pub. No.: US 2008/0090275 A1
(43) Pub. Date: Apr. 17, 2008

(54) Method of concentrating and disrupting cells or viruses

(75) Inventors: Yoon-kyoung Cho, Yongin-si (KR); Jeong-gun Lee, Yongin-si (KR); Jong-myeon Park, Yongin-si (KR); Young-sun Lee, Yongin-si (KR); Hyo-yeon Lee, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD., Suwon-si (KR)

(21) Appl. No.: 11/783,351

(22) Filed: Apr. 9, 2007

(30) Foreign Application Priority Data
Apr. 7, 2006 (KR) 10-2006-0031929
Dec. 12, 2006 (KR) 10-2006-0126412

(51) Int. Cl. C12N 13/00

(52) U.S. Cl. 435/173.9

(57) ABSTRACT

Provided is a method for concentrating and disrupting cells or viruses, the method employs magnetic particles which are capable of binding the cells or viruses to capture the cells or viruses and a laser to disrupt the cells or viruses. Since the same particles are used in target cell separation and cell lysis, there is no need to add additional particles in a laser lysis process, and thus integration of a target cell separation, concentration, purification, and nucleic acid extraction process is easy.

### CROSS-REFERENCE TO RELATED PATENT APPLICATION

This application claims the benefit of Korean Patent Application No. 10-2006-0031929, filed on Apr. 7, 2006, and No. 10-2006-0126412, filed on 12 Dec. 2006, in the Korean Intellectual Property Office, the disclosure of which is incorporated herein in its entirety by reference.

### BACKGROUND OF THE INVENTION

1. Field of the Invention

The present invention relates to a method for target cell separation and rapid nucleic acid isolation.

2. Description of the Related Art

Generally, molecular diagnosis of specific pathogenic bacteria is performed in four steps: cell lysis, DNA isolation, DNA amplification and DNA detection.

Efficient extraction of DNA from cells is required in many applications and is essential in the molecular diagnosis, in particular, identification and quantification of pathogenic bacteria. Molecular diagnosis is generally performed by DNA amplification after DNA extraction. DNA amplification includes a polymerase chain reaction (PCR), ligase chain reaction, stranded-displacement amplification, nucleic acid-based amplification, repair chain reaction, helicase chain reaction, QB replicase amplification, and ligation activated transcription.

Methods of isolating DNA from cells are performed using materials that have a proclivity of binding to DNA. Examples of materials for DNA isolation include silica, glass fiber, anion exchange resin and magnetic beads (Rudi, K. et al., Biotechniqures 22, 506-511 (1997); and Deggerdal, A. et al., Biotechniqures 22, 554-557 (1997)). To avoid manual operation and remove operator's errors, several automatic machines have been developed for high-throughput DNA extraction.

Cell lysis is conventionally performed using a mechanical, chemical, thermal, electrical, ultrasonic or microwave method (Michael T. Taylor et al., Anal. Chem., 73, 492-496 (2001)).

A laser has many advantages in the disruption of cells and can be readily applied to Lab-On-a-Chip (LOC) (Huaina Li et al., Anal Chem, 73, 4625-4631 (2001)).

U.S. Patent Publication No. 2003/96429 A1 discloses a laser-induced cell lysis system. However, this publication discloses a cell lysis system using only a laser, but does not teach or suggest that cell lysis is performed using magnetic beads and a laser.

Generally, it is possible to separate target cells from complex fluids such as whole blood by using antibody-coated magnetic beads that are commercially available. However, subsequent processes of nucleic acid extraction are complicated, time-consuming, and require various kinds of buffer solutions and thus the cell separation using magnetic beads is not suitable for a Lab-On-a-Chip.

Recently the inventors of the present invention reported a novel cell lysis method, laser-irradiated bead system (LIBS) (Lee et al., Lab Chip, Vol. 6, pp. 886-895, 2006), the disclosure of which is incorporated herein in its entirety by reference. It was observed that addition of beads to the pathogen containing solution accelerated the heating speed. Therefore, DNAs from various types of pathogens including both Gram-negative and Gram positive bacteria and hepatitis B viruses were effectively extracted by simply applying 40 seconds of laser (808 nm, 1.0 W) irradiation. However, this method cannot be used to extract DNAs from raw samples such as whole blood or to concentrate cells or extracted DNAs.

Therefore, there has been a need to develop an improved method to extract nucleic acids from raw samples and/or concentrate extracted nucleic acids or cells.

### SUMMARY OF THE INVENTION

The present invention provides a method for target cell separation/concentration and rapid nucleic acid isolation using antibody or affinity binding-based magnetic beads.

According to an aspect of the present invention, there is provided a method of concentrating and disrupting target, cells or viruses, the method including: bringing a particle into contact with a cell-containing or virus-containing sample to form cell-particle or virus-particle complexes, wherein the particle is coupled to a substance which is capable of binding to the target cells or viruses; and irradiating an electromagnetic wave from an external energy source to the sample containing the complexes to disrupt the cells or viruses to release nucleic acid materials from the cells or viruses.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram explaining a principle of a method of concentrating and disrupting cells or viruses according to an exemplary embodiment of the present invention;

FIG. 2 is a schematic diagram of a cell lysis system using a laser and micro particles in a microchip;

FIG. 3A is a graph showing results of real time PCR using a conventional laser-irradiated bead system (LIBS) method, which does not include cell concentration, and a TS-LIBS method according to an exemplary embodiment of the present invention including cell concentration, and FIG. 3B represents real time PCR results of FIG. 3A in terms of Rn values; and

FIGS. 4A through 4C are graphs showing effects of the types of beads, bead concentration and binding time with respect to the real-time PCR results, respectively, FIG. 4D is a graph showing virus capture efficiency of antibody-conjugated beads according to the binding number of viruses, and FIG. 4E is a graph showing virus DNA isolation efficiency using a TS-LIBS according to an exemplary embodiment of the present invention and a commercially available kit (Qiagen, QIAamp MinElute virus vacuum kit, 57714), respectively.

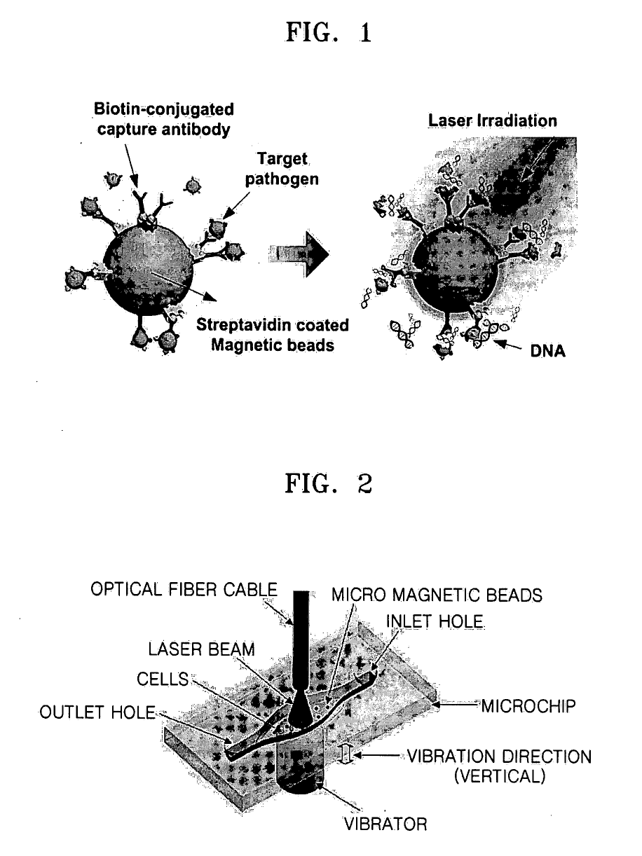

### DETAILED DESCRIPTION OF THE INVENTION

Hereinafter, the present invention will be described in more detail by explaining embodiments of the invention with reference to the attached drawings.

In the method according to an embodiment of the present invention, target cells or viruses are separated and concentrated from complex fluids such as whole blood, saliva, urine and the like in the form of a particle-cell (or virus) complex, and then a laser is directly irradiated thereto, and finally nucleic acids of interests are isolated. The particle is coupled with a substance that specifically or non-specifically binds to target cells or viruses in the sample fluids.

The formation of the particle-cell (or virus) complex allows a simultaneous separation and concentration of the target cells or viruses, improving the separation of target cells or viruses. A laser is irradiated to the complex to lysis the target cells or virus, from which genetic materials of interests are rapidly isolated. The method according to an embodiment of the present invention may be integrated into a Lab-On-a-Chip system.

The substance coupled to a particle and specifically binds to target cells or viruses may include, but is not limited to, an antibody. Particles which are suitable for use in separation target biological materials such as cells or viruses from biological sample fluids are well known in the art.

For example, when an antibody that specifically binds to biotin-bound cells or viruses is, reacted with particles such as beads to which streptavidin is attached, the antibody is bound to the beads by a specific affinity binding between streptavidin and biotin. Then, a cell-containing or virus-containing sample is contacted with them. As a result, the antibody that can specifically bind to target cells or viruses is bound to the cells or viruses so that the target cells or viruses are concentrated. Therefore, if various kinds of antibodies are used, desired cells or viruses can be concentrated. Conventionally, cell lysis of concentrated cells or viruses is performed using a Boom method or the like, but this method is complicated and time-consuming. However, in the method according to the current embodiment of the present invention, since the particle-cell (or virus) complexes, which are formed by a separation process, are subjected to cell disruption, nucleic acids can be rapidly and economically isolated. In particular, when a laser is irradiated to particle-cell (or virus) complexes, the cells or viruses can be rapidly disrupted by a laser ablation phenomenon.

FIG. 1 is a schematic diagram explaining a principle of a method of concentrating and disrupting cells or viruses according to an exemplary embodiment of the present invention. In FIG. 1, magnetic beads having a particular antibody are depicted, but it should be understood that the particles which can be used in the present invention are not limited to those. As shown in FIG. 1, beads conjugated with an antibody, which has specific affinity to target cells or viruses (e.g., pathogens), are mixed with a sample solution. Target cells or viruses are selectively captured on the beads and the waste materials such as plasma residue are washed away. Simple irradiation of laser (e.g., 808 nm, 1.5 W) for about 30 seconds could effectively extract PCR-ready DNA from captured target pathogens. Throughout the specification, descriptions are provided with respect to beads as a representative example of a particle suitable for use in the present application, but the particle is not limited to beads.

In the method according to the current embodiment of the present invention, a surface of the beads can be treated with an antibody or metal oxide that has an affinity to target cells or viruses. The surface of the beads may be treated with an antibody that can specifically bind to cells or viruses. Since only target cells or viruses are selectively captured by the beads through antibody-cell (or virus) binding and then concentrated, the method is useful in case of detecting cells or viruses having a very low concentration. Beads to which an antibody that can specifically bind to cells or viruses is bound is commercially available from Invitrogen, Qiagen and the like. Examples thereof include, but are not limited to, Dynabeads® Genomic DNA Blood (Invitrogen), Dynabeads® anti-E. coli O157 (Invitrogen), CELLection™ Biotin Binder Kit (Invitrogen), MagAttract Virus Min M48 Kit (Qiagen) and the like. Using the beads to which the specific antibody is bound, Diphtheria toxin, Enterococcus faecium, Helicobacter pylori, HBV, HCV, HIV, Influenza A, Influenza B, Listeria, Mycoplasma pneumoniae, Pseudomonas sp., Rubella virus, Rotavirus and the like can be separated.

The method according to the current embodiment of the present invention can further comprise performing a PCR using nucleic acids isolated after the disruption of the cells or viruses. As used herein, the “PCR” refers to a “polymerase chain reaction” and is a method for amplifying a target nucleic acid from a primer pair specifically binding with the target nucleic acid, using a polymerase. PCR is well known in the art and can be performed using a commercially available kit. The amplification of a target nucleic acid can also be performed using an appropriate method known in the art, e.g., ligase chain reaction, nucleic acid sequence-based amplification, transcription-based amplification system, strand displacement amplification, Qβ replicase, or other nucleic acid amplification methods, in addition to PCR. In one embodiment, real-time PCR may be used.

In the method according to the current embodiment of the present invention, a process of vibrating the beads can be additionally included in a process of concentrating the cells or viruses. When the beads are vibrated, the beads are more likely to contact cells or viruses compared with when the beads are not vibrated, and thus cells or viruses can be more efficiently concentrated onto the beads. Beads can be vibrated through a vibrator such as a sonicator, a vibrator using a magnetic field, a vibrator using an electric field, or a mechanical vibrator such as a vortex or a piezoelectric material.

In the method according to the current embodiment of the present invention, the metal oxide can be Al₂O₃, TiO₂, SiO₂, Ta₂O₃, Fe₂O₃, Fe₃O₄, and HfO2, but is not limited thereto. In one embodiment, the metal oxide may be Al₂O₃ or TiO₂, and preferably Al₂O₃. Deposition of the metal oxide can be performed using a physical vapor deposition (PVD) method, an atomic layer deposition (ALD) method, a sol-gel method or the like. A method of depositing metal oxide on a surface of beads is a known technique, and generally is performed using a PVD method, an ALD method, a sol-gel method or the like.

PVD is a method that is used in thin film formation, and has recently been favored as a means of surface curing, since a thin film can be relatively simply obtained by low temperature treatment that cannot be performed using other methods. Examples of PVD methods include an evaporation deposition method that does not use ions, a sputtering method that uses ions, an ion plating method, an ion implantation method, an ion beam mixing method and the like. In an ALD method, molecules are absorbed into a wafer surface using a chemically sticking property and then substituted. Since absorption and substitution are alternatively performed, ultrafine layer-by ultrafine layer deposition is possible, and an oxide and a metal thin film can be stacked as thin as possible. The sol-gel method is a method of preparing a metal oxide having a colloid form through hydrolysis reaction of a metal halide or alkoxide and a representative method of preparing a coating solution of titanium dioxide (TiO₂).

Once target cells or viruses are bound to beads to form bead-cell (or virus) complexes, a laser is irradiated into a solution containing the complexes and the beads cause an ablation by the laser, and thus shockwaves, vapor pressure and heat are transferred to the cell surface. At this time, physical shocks are also applied to the cell surface. The beads heated by the laser raise the temperature of the aqueous solution and directly disrupt the cells. The beads in the aqueous solution not only act as a heat conductor but apply thermal, mechanical and physical shocks to the cell surface, thereby efficiently disrupting the cell surface. When only a laser is used, efficient cell lysis does not occur. From an experiment using E. coli in a very clear solution, it is confirmed that when just a laser was irradiated, low cell lysis efficiency was obtained. A concentration of DNA measured after irradiating a laser for 150 seconds was 3.77 ng/μl because laser energy was not effectively transferred to cells, but a concentration of DNA measured after boiling cells at 95° C. for 5 minutes was 6.15 ng/μl.

The rapid cell lysis using a laser and beads is performed by the application of heat and laser to a liquid medium. The laser in combination with the micro beads converts the heat source into physical and mechanical shocks of highly heated beads to improve cell lysis. Currently, small size, high power laser diodes are rapidly being developed and a very small-sized cell lysis apparatus using such as high power laser diodes will be available to be installed on a Lap-on-a-Chip (LOC). Moreover, the application of the laser can be focused on a specific region on a chip directly or by means of an optical fiber, mirror or lens.

An advantage of using the beads is an omission of DNA isolation steps because the cell lysis by means of the micro beads and laser results in the denaturation of proteins: The denatured proteins and cell debris are attached to the beads, which may be removed by gravity or magnetic force. As a result, a detection limit is lowered and a DNA extraction time is significantly shortened due to an omission of one step in the DNA extraction process. Furthermore, polymerase chain reaction (PCR) analysis results are significantly improved due to an increase in the signal amplitude. The total time required for disrupting a cell using the micro beads and laser may be as short as only 40 seconds.

Laser ablation refers to a phenomenon that occurs in materials exposed to a laser beam. Laser ablation rapidly raises the temperature of a material surface from several hundred to several thousand degrees. If the temperature of the material surface is raised to the evaporation point of the material or higher, the saturated vapor pressure on the surface rapidly increases according to an evaporation of the liquid phase material. The saturated vapor pressure is expressed as a function of temperature by the Clausius-Clapeyron equation, and is usually raised to several tens of atmospheres or more in the case of a high power pulse laser process. Pressure applied to a material surface by vapor is referred to as “repulsive pressure” and the magnitude of the repulsive pressure is about 0.56 Pₛₐₜ where Pₛₐₜ denotes a vapor pressure.

A shockwave is generated in a process using a laser with very large instantaneous intensity, such as a pulse laser. The vapor generated on the surface of a material heated to its evaporation point or higher for a short time ranging from several nanoseconds to several tens of nanoseconds is increased to a pressure of several atmospheres to several tens of atmospheres and forms shockwaves while expanding into the surrounding air. Due to the very high pressure, the expanding vapor applies about 0.56 Pₛ (where Pₛ denotes a saturated vapor pressure in the surface) to a material.

In the method according to the current embodiment of the present invention, cells or viruses in a sample may be disrupted by irradiating an electromagnetic wave to the sample which contains particle-cell or particle-virus complexes. The electromagnetic wave is supplied by an external energy source. In one exemplary embodiment, the electromagnetic wave is a laser. The laser can be a pulse laser or continuous wave (CW) laser. If the laser power is too low, laser ablation cannot effectively occur. The laser power is 10 mW or more for the CW laser and 1 mJ/pulse or more for the pulse laser. In one embodiment, the laser power for the pulse laser is 3 mJ/pulse or more and 100 mW or more for the CW laser. This is because when the laser power for the CW laser is less than 10 mW and the laser power for the pulse laser is less than 1 mJ/pulse, sufficient energy to disrupt the cells is pot transferred.

In the method according to the current embodiment of the present invention, the laser should be generated in a specific wavelength range where beads absorb the energy of the laser. The laser may be generated in the wavelength range of 400 nm or more, and preferably in the wavelength range from 750 nm to 1,300 nm. This is because DNA is denatured or damaged at a wavelength less than 400 nm. The laser can also be generated in at least one wavelength range. That is, the laser can have one wavelength or at least two different wavelengths within the above range.

In the method according to the current embodiment of the present invention, the diameter of the beads is from 5 nm to 1,000 μm. In one embodiment, the diameter is from 1 μm to 50 μm. When the diameter of the beads is less than 5 nm, physical and mechanical shocks are insufficient to cause cell lysis. When the diameter of the beads is greater than 1,000 μm, it is not suitable for Lab-On-a-Chip (LOC). The beads can also be a mixture of beads with at least two sizes. That is, the beads can be all the same size or be a mixture of beads with different sizes.

In the method according to the current embodiment of the present invention, the beads can be any magnetized material. In particular, the beads may include at least one material selected from the group consisting of ferromagnetic Fe, Ni, Cr and oxides thereof.

In the method according to the current embodiment of the present invention, the beads can be polymers, organic materials, silicon or glass coated with a ferromagnetic metal.

In the method according to the current embodiment of the present invention, a solution containing beads may have a pH of 6-9. When the pH of the solution is beyond this range, efficiency of DNA amplification decreases after cell lysis.

In the method according to the current embodiment of the present invention, the solution can be selected from the group consisting of saliva, urine, blood, serum and cell culture. The solution can be any solution having nucleic acids, such as animal cells, plant cells, bacteria, viruses, phage and the like.

Hereinafter, the present invention will be described in further detail with reference to the following examples. These examples are for illustrative purposes only and are not intended to limit the scope of the present invention.

### EXAMPLE 1

### E. coli Cell Lysis Using the Method of the Present Invention

### 1) E. coli Cell Preparation

E. coli K12 (ATCC 25404) as a bacterial cell was cultured in ATCC media 294, and then the bacterial cells were harvested by centrifugation and washed twice with 3 ml of phosphate-buffered saline (PBS). Subsequently the bacterial cells were resuspended in PBS (cell concentration: 1×10⁹ cells/ml). The obtained cells were diluted in PBS to have a final cell concentration of 1×10⁷ cells/ml.

### 2) Bead Washing

DYNABEADS® M-280 Streptavidin (INVITROGEN) having a diameter of 2.8 μm in which streptavidin was attached thereto was used as a bead, and a bead solution was mixed to prepare a homogeneous solution. 100 μl of the prepared solution was placed in a tube, and left sit for two minutes in a magnet. Supernatant of the resulting solution was removed with a pipette. The tube was taken out of the magnet, and 100 μl of a buffer solution 1 (PBS containing 0.1% of BSA, pH 7.4) was added to the solution in the tube and mixed with it. Again, the tube was placed in the magnet and left for two minutes. Supernatant of the resulting solution was removed with a pipette. The tube was taken out of the magnet, and 100 μl of a buffer solution 1 (PBS containing 0.1% BSA, pH 7.4) was added to the solution in the tube and mixed with it.

### 3) Pre-Coating of Beads Using Antibody

10 μl of E. coli antibody (ViroStat, ME, 1007 biotin conjugate, 4-5 mg/ml) was placed in the prepared bead solution and mixed with it. The solution was mixed many times with the tube inverting, and the mixture was incubated at a room temperature for 30 minutes. Beads were collected for 1 minute using a magnet, and supernatant of the resulting solution was removed. 1 ml of a washing buffer solution (PBS containing 1% BSA, pH 7.4) was added thereto and mixed many times with the tube inverting. Beads were collected for 1 minute using a magnet, and supernatant of the resulting solution was removed. 100 μl of a buffer solution 1 (PBS containing 0.1% BSA, pH 7.4) was added thereto.

### 4) E. coli Cell Separation

100 μl of the E. coli K12 cell prepared in Example 1-(1) and 100 μl of a bead solution to which the E. coli antibody was bound prepared in Example 1-(3) were mixed. By mixing them many times with the tube inverting, the mixture was incubated at a room temperature for 20 minutes. Beads were collected for 1 minute using a magnet, and supernatant of the resulting solution was removed. 100 μl of a washing buffer solution was added thereto and mixed many time with the tube inverting. Beads were collected using a magnet, and supernatant of the resulting solution was removed. 4 μl of a buffer solution 1 (PBS, pH 7.4) was added thereto.

### 5) Cell Lysis

4 μl of E. coli separated in the above examples was injected to a vial located in a vial guide (AMITECH, Korea). 808 nm, 1 W laser beam (HLU25F100-808, LIMO, Germany) was applied to the mixture for 30 seconds for disrupting cells for a specific time in individual experiments while stirring the vial by vortexing (Lee et al., Lab Chip, Vol. 6, pp. 886-895, 2006).

### EXAMPLE 2

### Bacteria Cell Capture Efficiency

Recently, Lee et al. (Lee et al., Lab Chip, Vol. 6, pp. 886-895, 2006) reported a rapid DNA extraction method using laser-irradiated bead system (LIBS). By adding beads to a cell solution and applying 40 seconds of laser (808 nm, 1.0 W) irradiation, DNA extraction from both Gram-negative and Grampositive bacteria and hepatitis B viruses were demonstrated. Even though the carboxylated surface of the beads was proven to be effective in adsorbing proteins that could be an inhibitor in the following step of PCR, raw samples such as whole blood could not be directly used. Furthermore, concentration of dilute sample was not possible.

In the present Example 2, it was confirmed that target specific separation and concentration could be possible using antibody conjugated beads. Therefore, the beads are used for dual purposes. First, the beads are modified with pathogen specific antibodies and thus act as a mediator for a pathogen specific cell separation and concentration. Second, the beads act as microheaters for rapid heat transfer. The 808 nm laser is not absorbed by water molecules but effectively absorbed by the beads dispersed in solution. As a result, the heating speed is ultra fast and the cell lysis step could be dramatically shortened. Furthermore, large volume of lysis buffer is not necessary, which is another favorite characteristic for the miniaturization.

Capture efficiency of E. coli K12 cell with respect to DYNABEADS® M-280 Streptavidin to which E. coli antibody (ViroStat: 1007 biotin conjugate, 4-5 mg/ml) was bound prepared in Example 1-(3) was determined. The number of E. coli cells was counted using 3M colony count paper. Three kinds of E. coli K12 concentration, that is, 1.0E+05 cells/μl, 1.0E+04 cells/μl and 1.0E+03 cells/μl were used. Capture efficiency of E. coli K12 cell bound to the beads is shown in Table 1 below.

TABLE I

| E. coli cell | Capture | |
|---|---|---|
| Concentration | Efficiency (%) | CV (%) |
| (cell/μl) | (I − B − W)/I*100 | (N = 4) |
| 1.0E+05 | 93.4 | 0.2 |
| 1.0E+04 | 87.0 | 0.9 |
| 1.0E+03 | 92.5 | 1.6 |

In Table 1, I refers to the number of E. coli cells in an injected solution, B refers to the number of E. coli cells remaining in a binding solution, and W represents the number of E. coli cells remaining in a washing buffer solution. Capture Efficiency is represented by the formula ((I—B—W)/I*100). As can be seen in Table 1, E. coli cell capture efficiency is as high as about 90%. Accordingly, when the method according to the current embodiment of the present invention is used, E. coli can be captured at a very high efficiency, and thus the method according to the current embodiment of the present invention is very useful for samples having a very low cell concentration.

### EXAMPLE 3

### Effects of PCR Inhibition by Beads

When 250 mg/ml of DYNABEADS® M-280 Streptavidin according to an embodiment of the present invention was used, capture efficiency was about 90% (see Example 2). In an experiment of cell capture using beads, 100 μl of a bead having a concentration of 10-mg/ml was reacted with 100 μl of E. coli, and the product was washed. Then the resulting product was eluted in 4 μl of an elution solution. As a result, the concentration of the bead was 250 mg/ml. The concentration was 25 times as high as that of DYNABEADS® MyOne™ Carboxylic Acid (DYNAL, Norway)(10 mg/ml) in which the bead is used as a material that generates cell lysis without having a cell concentration function (Lee et al., Lab Chip, Vol. 6, pp. 886-895, 2006). Therefore, whether PCR inhibition occurs even when the amount of beads is increased was determined.

As in Example 2, 6×10⁹ cells/ml, 6×10⁸ cells/ml and 6×10⁷ cells/ml were used as a concentration of E. coli K12. Cells in a cell and bead mixture comprising 10% of beads and 90% of a cell solution were lysed using a laser, and then TaqMan real time PCR was performed. PCR was performed by completely denaturing DNAs in a PCR mixture (75 mM Tris-HCl (pH 9.0), 15 mM (NH₄)₂SO₄, 5 mM MgCl₂, 1 mg/ml BSA, 250 μM dNTP mixture, 1 μM PCR primer (SEQ ID NO: 1 and SEQ ID NO: 2) and 0.4 μM TaqMan probe FAM-5′-TGTATGAAGAAGGCTTCGGGTTGTAAAGTACTTTCAGCGGGGAGGA AGGGAGTAAAGTTAATACCTTT-3′-TAMRA: SEQ ID NO: 3) using GENESPECTOR® Micro PCR (SAIT, Korea) at a temperature of 95° C. for 1 minute, and then 40 cycles (10 seconds at 95° C., 10 seconds at 50° C. and 10 seconds at 72° C.) were performed. Results of measuring the crossing point (Cp) of a PCR product are shown in Table 2. Cp refers to a cycle number in which a detectable fluorescence signal in a real time PCR occurs. That is, as an initial DNA concentration is higher, it is possible for a fluorescence signal to be detected at a low Cp, and the lower the initial DNA concentration is, the higher Cp is. Cp is also related to DNA purification, that is, the higher DNA purity is, the lower Cp is, and the lower DNA purity is, the higher Cp is. Accordingly, a smaller value of Cp indicates that DNA in a solution is more purified.

TABLE 2

| Injected cell | Myone bead | M-280 Streptavidine bead (mg/ml) | | | |
|---|---|---|---|---|---|
| (cell/ml) | (10.0 mg/ml) | 12.5 | 25.0 | 50.0 | 250.0 |
| 6.00E+09 | 22.90 | 22.36 | 22.52 | 22.04 | 22.53 |
| 6.00E+08 | 24.21 | 24.53 | 23.87 | 24.22 | 23.67 |
| 6.00E+07 | 28.36 | 25.78 | 25.11 | 25.99 | 27.40 |

As can be seen in Table 2, even when the amount of beads used in the present invention is 25 times that of DYNABEADS® MyOne™ Carboxylic Acid (DYNAL, Norway), there was little impact on PCR. Therefore, when the method according to the current embodiment of the present invention is used, cell capture efficiency is as high as about 90%, and beads used in nucleic acid binding and lysis barely influence nucleic acid amplification of DNA released from the captured cells, and thus beads according to an embodiment of the present invention are very useful for capturing cells and isolating nucleic acids.

### EXAMPLE 4

### Effects of Cell Concentration Using the Method According to the Current Embodiment of the Present Invention

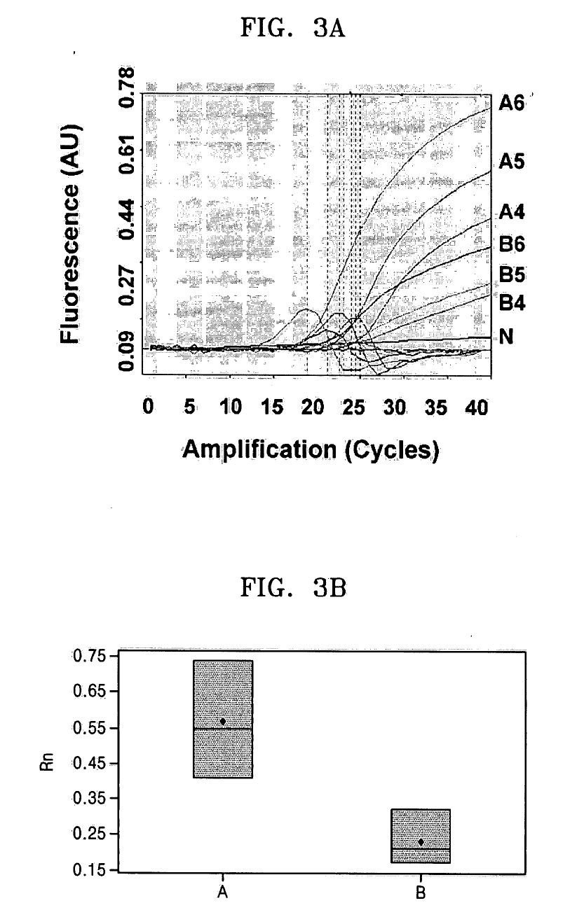

Cell capture efficiency of the method according to the current embodiment of the present invention was compared with a conventional method of mixing a micro bead (DYNABEADS® MyOne™ Carboxylic Acid (DYNAL, Norway)). An experiment similar to Example 1 was performed. The results of measuring Cp and Rn of a real time PCR product are shown in FIGS. 3A and 3B. FIG. 3A is a graph showing results of performing a real time PCR using laser-irradiated bead system (LIBS), which does not include a cell concentration function, and target separation and laser-irradiated bead system (TS-LIBS) according to the current embodiment of the present invention, which includes a cell concentration function. In FIG. 3A, TS-LIBS according to the current embodiment of the present invention including a cell concentration function is used for “A6”, “A5” and “A4,” as indicated on the right side of the graph, and each of 10⁹ cells/ml, 10⁸ cells/ml and 10⁷ cells/ml is used for “A6”, “A5” and “A4”. LIBS without a cell concentration function is used for “B6”, “B5” and “B4,” as indicated on the right side of the graph, and each of 10⁹ cells/ml, 10⁸ cells/ml and 10⁷ cells/ml is used for them. In addition, a negative control is represented as “N”. In FIG. 3B, real time PCR results of FIG. 3A are represented by Rn. In FIG. 3B, A refers to the PCR results using TS-LIBS according to the current embodiment of the present invention including a cell concentration function, and B represents the PCR; results using LIBS which does not have a cell concentration function. Cp is related to an initial PCR template concentration, and the lower the Cp is, the higher the initial concentration is. If the PCR efficiency is 100%, ΔCp 3.3 denotes 10 times difference of an initial concentration. Rn is related to a concentration of a PCR product, and the higher Rn is, the higher the concentration of a PCR product is. Results of FIG. 3 are shown in Table 3 below.

TABLE 3

| | Cell | | | | |
|---|---|---|---|---|---|
| | concentration | | | | |
| Methods | (cell/ml) | Cp | Rn | ΔCp | ΔRn |
| LIBS | 1.00E+09 | 21.25 | 0.32 | | |
| (no concentration | 1.00E+08 | 23.04 | 0.21 | | |
| function) | 1.00E+07 | 24.96 | 0.17 | | |
| TS-LIBS | 1.00E+09 | 18.88 | 0.74 | 2.37 | 0.43 |
| (concentration | 1.00E+08 | 22.57 | 0.55 | 0.47 | 0.34 |
| function) | 1.00E+07 | 24.51 | 0.41 | 0.45 | 0.23 |

As can be seen in FIG. 3 and Table 3, compared with a conventional method of mixing a micro bead (Dynabeads® MyOne™ Carboxylic Acid (DYNAL, Norway)), cell capture efficiency of the method according to the current embodiment of the present invention is about 2-7 times higher than that of the conventional method. Accordingly, when the method according to the current embodiment of the present invention is used, cell concentration efficiency is very high compared with the conventional, method.

### EXAMPLE 5

### Effects of Cell Concentration from Whole Blood Using the Method According to the Current Embodiment of the Present Invention

To selectively separate/concentrate E. coli from E. coli spiked in human whole blood, not in a phosphate-buffered saline, the method of Example 1 was performed. Results of real time PCR performed after separating/concentrating E. coli spiked in a phosphate buffer solution or whole blood, using LIBS without a cell concentration function and TS-LIBS including a cell concentration function are shown in Table 4 below. In Table 4, X denotes that a PCR product was not obtained.

TABLE 4

| | | | PCR product |
|---|---|---|---|
| Sample types | Method | Cp | concentration (ng/ml) |
| PBS buffer | TS-LIBS | 19.40 | 20.3 |
| Whole blood | TS-LIBS | 19.43 | 17.9 |
| PBS buffer | LIBS | 21.05 | 15.3 |
| Whole blood | LIBS | X | X |

As can be seen in Table 4, when TS-LIBS was employed, the cell capture efficiency and PCR results obtained from a whole blood sample and from a phosphate-buffered saline were similar. When a sample is a phosphate-buffered saline, it can be seen that Ct of TS-LIBS test has 1.65 lower value than that of LIBS test. An initial HBV concentration calculated using a calibration curve showed that DNA concentration increased by about 3.4 times when target separation and concentration according to the method of the present invention were further added (data not shown).

In addition, when LIBS was used, PCR with respect to E. coli cells in a PBS buffer was efficiently performed. However, no PCR product was obtained from E. coli cells spiked in whole blood.

Therefore, even when real samples such as blood, saliva, urine and the like that cannot be directly used in PCR are used, cell capture and PCR can be efficiently performed using TS-LIBS according to the method of the present invention.

### EXAMPLE 6

### Virus Concentration Using the Method of the Present Invention and DNA Extraction Using Laser Irradiation

DNA isolation from whole blood spiked with HBV was conducted using TS-LIBS. In order to find the optimum condition of the TS-LIBS method, the diameter of a bead, surface property and the concentration of the beads, the binding time, the volume of a washing buffer and the number of the washing steps have been investigated.

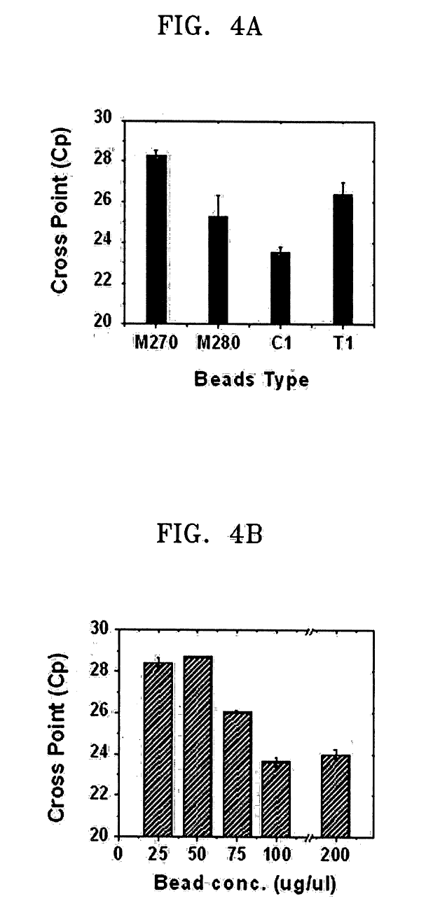

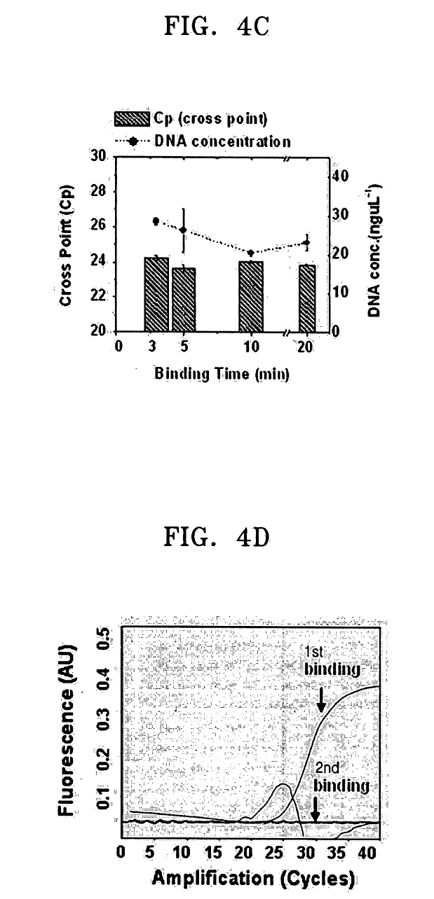

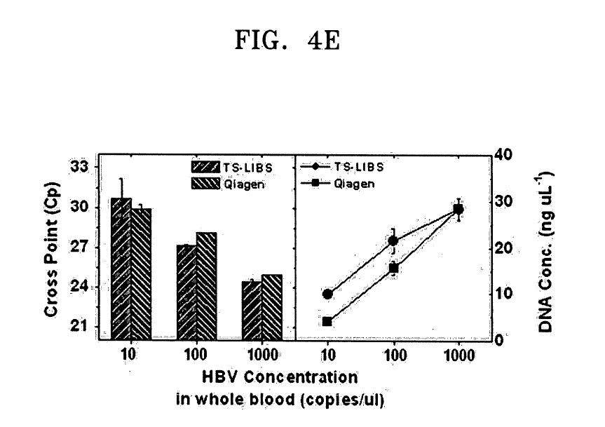

As shown in FIG. 4A, the C1 type of the beads (Dynabeads Myone Streptavidin C1 (Dynal), diameter: 1 μm, binding capacity of biotinylated Ig: 15-20 μg/mg, hydrophilic, carboxylic acid beads) showed the best results compared to other kinds of beads with larger sizes; e.g. M-280 (Dynabeads M-280 Streptavidin (Dynal), diameter: 2.8 μm, binding capacity of biotinylated Ig: 5-10 μg/mg, hydrophobic, tosyl activated beads) and M-270 (Dynabeads M-270 Streptavidin (Dynal), diameter: 2.8 μm, binding capacity of biotinylated Ig: 5-10 μg/mg, hydrophilic, carboxylic acid beads) or hydrophobic and tosyl activated beads; e.g. T1 (Dynabeads Dynabeads Myone Streptavidin T1 (Dynal), diameter: 1 μm, binding capacity of biotinylated Ig: 40-50 μg/mg, hydrophobic, tosyl activated beads).

Since the same mass concentration of the beads were used, the surface area of the beads with smaller sizes was larger and thus binding capacity of the biotin labeled antibody was larger. In addition, as previously reported, carboxylated beads showed superior protein removal function compared to other amine modified or polystyrene beads.

As shown in FIG. 4B, the larger concentration of the beads resulted in better PCR products up to 100 μg/μl. Since 100 μl of C1 beads (10 mg/ml) was used and the final lysis chamber volume was 10 μl, the bead concentration in lysis chamber was 100 μg/μl.

Commercial sample preparation kits using beads usually recommend the incubation time of 20 minutes for the binding of antibodies on the bead surface. However, as shown in FIG. 4C, when the binding time varied in the range of 3-20 minutes, the lysis efficiency was not affected much and thus the binding time of 3 minutes were used thereafter.

The measurement of the capture efficiency was not trivial for the virus sample because the virus culture was not possible. Instead, the antibody conjugated beads to the virus solution was added twice and DNA was amplified from each step. As shown in FIG. 4D, most of the viruses were captured in the first binding step, and no PCR amplicon was obtained in the second binding experiment.

The DNA isolation efficiency of TS-LIBS was compared with a commercial kit (Qiagen, QIAamp MinElute virus vacuum kit, 57714). MinElute virus vacuum kit requires 500 μl of serum or plasma sample and takes over one hour with many manual steps of adding various buffers. The TS-LIBS of the present invention uses only 30 μl of serum or plasma sample and takes 12 minutes with only one step.

Although the initial sample volume requirements are different, the same concentration factor, i.e. the ratio of the initial plasma sample volume to the final volume of the DNA solution was used for the comparison purpose. For the Qiagen preparation kit, 100 μl of plasma sample was mixed with 400 μl of PBS buffer and the final elution volume was 33 μl. Since 301 of plasma was used and the final volume was 10 μl in TS-LIBS, the concentration factor was the same.

As shown in FIG. 4E, the real-time PCR results obtained by the TS-LIBS method were as good as those obtained by a commercially available kit (Qiagen, QIAamp MinElute virus vacuum kit, 57714). The limit of the detection was 10 copies/μl for both of the methods. It is noteworthy that the cut-off range for the HBV DNA test in current clinical diagnostics is 100 copies/μl.

As described above, according to the method of the present invention, since the same commercially available beads are used in target cell separation and cell lysis, beads need not to be additionally added in a laser lysis process, and thus integration of a target cell separation, concentration, purification, and nucleic acid extraction process is easy. Also, in a conventional method, blood acts as an inhibitor for PCR, so that blood samples cannot be directly used. Thus, target cells in blood can be lysed and processed by PCR only after cell separation and purification. However, according to the present invention, target cells in blood can be easily separated and concentrated using beads for target cell separation, and nucleic acid extraction can be easily integrated by irradiating a laser in the same beads.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of concentrating and disrupting target cells or viruses, the method comprising:
bringing a particle into contact with a sample containing the cells or viruses to form cell-particle or virus-particle complexes, wherein the particle is capable of binding to the target cells or viruses; and
irradiating an electromagnetic wave from an external energy source to the sample containing the complexes to disrupt the cells or viruses to release nucleic acid materials from the cells or viruses.

2. The method of claim 1, wherein the particle is a bead, of which surfaces are treated with an antibody or metal oxide that has an affinity to the target cells or viruses.

3. The method of claim 1, further comprising performing PCR using the nucleic acid materials isolated from the cells or viruses.

4. The method of claim 1, wherein the bringing the particle into contact with the sample further comprises vibrating the particles.

5. The method of claim 2, wherein the metal oxide is selected from the group consisting of Al₂O₃, TiO₂, SiO₂, Ta₂O₃, Fe₂O₃, Fe₃O₄ and HfO2.

6. The method of claim 1, wherein the electromagnetic wave is a laser.

7. The method of claim 6, wherein the laser is a pulse laser or a continuous wave (CW) laser.

8. The method of claim 7, wherein the power of the pulse laser is more than 1 mJ/pulse, and the power of the continuous wave (CW) laser is more than 10 mW.

9. The method of claim 8, wherein the power of the pulse laser is more than 3 mJ/pulse, and the power of the continuous wave (CW) laser is more than 100 mW.

10. The method of claim 6, wherein the laser is generated in a wavelength range of more than 400 nm.

11. The method of claim 10, wherein the laser is generated in a wavelength range of 750 nm to 1,300 nm.

12. The method of claim 10, wherein the laser is generated in at least one wavelength range.

13. The method of claim 1, wherein the particle has a size of 5 nm to 1,000 μm.

14. The method of claim 13, wherein the particle has a size of 1 μm to 50 μm.

15. The method of claim 13, wherein the particle is a mixture of particles having at least two sizes.

16. The method of claim 1, wherein the particle comprises at least one material selected from the group consisting of ferromagnetic Fe, Ni, Cr and oxides thereof.

17. The method of claim 1, wherein the particle is made of a polymer, organic material, silicon or glass and is coated with a ferromagnetic metal.

18. The method of claim 1, wherein a sample containing the particle-cell or particle-virus complexes has a pH of 6-9.

19. The method of claim 1, wherein the sample is selected from the group consisting of saliva, urine, blood, serum and a cell culture.
